(19) United States
(12) Patent Application Publication
(10) Pub. No.: US 2014/0056862 A1
(43) Pub. Date: Feb. 27, 2014

(54) NUTRITIONAL COMPOSITIONS HAVING ALPHA-HICA AND ALPHA-KETOGLUTARATE

(71) Applicants: Norman Alan Greenberg, New Hope, MN (US); Denis Breuille, Lausanne (CH); Zamzam Kabiry (Fariba) Roughead, Plymouth, MN (US); Doug Bolster, Lake in the Hills, IL (US); Jennifer Mager, St. Louis, MO (US)

(72) Inventors: Norman Alan Greenberg, New Hope, MN (US); Denis Breuille, Lausanne (CH); Zamzam Kabiry (Fariba) Roughead, Plymouth, MN (US); Doug Bolster, Lake in the Hills, IL (US); Jennifer Mager, St. Louis, MO (US)

(73) Assignee: Nestec S.A., Vevey (CH)

(21) Appl. No.: 14/112,746

(22) PCT Filed: Apr. 18, 2012
(86) PCT No.: PCT/EP12/57095

§ 371 Date: Oct. 18, 2013

Related U.S. Application Data
(60) Provisional application No. 61/476,345, filed on Apr. 18, 2011.

(51) Int. Cl. A61K 31/194; A61K 31/198; A61K 35/74; A61K 36/06; A23L 1/29; A61K 31/711; A61K 31/7105; A61K 36/064; A61K 45/06; A61K 31/19; A61K 31/205

(52) U.S. Cl. CPC A61K 31/194; A61K 31/19; A61K 31/198; A61K 35/741; A61K 35/747; A61K 36/06; A61K 31/205; A61K 31/711; A61K 31/7105; A61K 36/064; A61K 35/745; A61K 45/06; A23L 1/296
USPC 424/93.41; 514/574; 514/564; 424/93.4; 424/93.42; 424/93.44; 424/93.45; 424/93.5; 514/556; 514/565; 514/560; 514/44 R; 424/93.51; 424/94.1; 514/690; 514/21.9; 424/725; 514/474; 514/27; 424/702; 514/458

(57) ABSTRACT

Nutritional compositions for maximizing muscle protein synthesis while minimizing the catabolism of muscle proteins and methods of using same are provided. In this manner, the nutritional compositions may provide for retention of lean body mass, which helps to avoid loss of independence and functionality, as well as to improve quality of life especially in the elderly at risk of sarcopenia and frailty. The nutritional compositions include α-hydroxyisocaproic acid and α-ketoglutarate. The composition may include other functional ingredients such as, but not limited to whey protein including whey protein micelles, prebiotic fibers, citrulline, eicosapentaenoic acid, L-carnitine, nucleotides, and amino acids. Methods of administering such nutritional products to individuals in need of same are also provided.

### BACKGROUND

The present disclosure relates generally to health and nutrition. More specifically, the present disclosure relates to nutritional compositions having α-hydroxyisocaproic acid and derivatives and methods of using same.

There are many types of nutritional compositions currently on the market. Nutritional compositions can be targeted toward certain consumer types, for example, young, elderly, athletic, etc., based on the specific ingredients of the nutritional composition. For example, the elderly and individuals with certain illnesses can often times experience a reduction in lean body mass that is due, at least in part, to a reduction in muscle protein synthesis (such as sarconpenia), reduced intake, or increased demand due to illness or presence of inflammation. A reduction in lean body mass can lead to the loss of metabolic stability (glucose tolerance, insulin sensitivity), independence, functionality, and quality of life, as well as a decline in cognitive ability. These individuals, therefore, would benefit significantly by administration of a diet directed to maximizing the anabolism and minimizing the catabolism of muscle tissue. The diets could also provide further benefits to the individuals by combining into a nutritional composition different types of functional compounds, which provide different types of physiological advantages.

One goal of nutritional support, therefore, is to provide individuals requiring improved muscle performance and/or maintenance of lean body mass and muscle strength/power with nutritional compositions that provide physiological benefits with respect to same.

### SUMMARY

The present disclosure is directed to nutritional compositions having α-hydroxyisocaproic acid and an effective amount of α-ketoglutarate. Thus, in an embodiment, the nutritional compositions include an effective amount of α-hydroxyisocaproic acid and an effective amount of α-ketoglutarate.

In an embodiment, the α-hydroxyisocaproic acid is present in an amount from about 0.15 to about 10 g, preferably from about 2 g to about 10 g. The α-hydroxyisocaproic acid may also be present in an amount of about 0.5 g to about 5 g, more preferably from about 2 g to 5 g, most preferably about 1.5 g.

In an embodiment, the nutritional compositions include a source of ω-3 fatty acids, wherein the source of ω-3 fatty acids is selected from the group consisting of fish oil, krill, plant sources containing ω-3 fatty acids, flaxseed, canola oil, walnut, algae, or combinations thereof. The ω-3 fatty acids are selected from the group consisting of α-linolenic acid (“ALA”), stearidonic acid (SDA), docosahexaenoic acid (“DHA”), eicosapentaenoic acid (“EPA”), or combinations thereof.

In an embodiment, the nutritional compositions include at least one nucleotide selected from the group consisting of a subunit of deoxyribonucleic acid (“DNA”), a subunit of ribonucleic acid (“RNA”), polymeric forms of DNA and RNA, yeast RNA, or combinations thereof. The at least one nucleotide may be an exogenous nucleotide. The nucleotide may be provided in an amount of about 0.5 g to 3 g per day.

In an embodiment, the nutritional compositions include a phytonutrient selected from the group consisting of flavanoids, allied phenolic compounds, polyphenolic compounds, terpenoids, alkaloids, sulphur-containing compounds, or combinations thereof. The phytonutrient may be selected from the group consisting of carotenoids, plant sterols, quercetin, curcumin, limonin, or combinations thereof.

In an embodiment, the nutritional compositions include a source of protein. The source of protein may provide the nutritional composition with at least 10 g of high quality protein or to provide an amount of protein of at least 10 g per day. The source of protein may be selected from the group consisting of dairy based proteins, plant based proteins, animal based proteins, artificial proteins, or combinations thereof. The dairy based proteins may be selected from the group consisting of casein, micellar casein, caseinates, casein hydrolysate, whey, whey hydrolysates, whey concentrates, whey isolates, milk protein concentrate, milk protein isolate, or combinations thereof. The plant based proteins are selected from the group consisting of soy protein, pea protein, canola protein, wheat and fractionated wheat proteins, corn proteins, zein proteins, rice proteins, oat proteins, potato proteins, peanut proteins, green pea powder, green bean powder, spirulina, proteins derived from vegetables, beans, buckwheat, lentils, pulses, single cell proteins, or combinations thereof.

In an embodiment, the nutritional compositions include a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

In an embodiment, the nutritional compositions include a probiotic selected from the group consisting of Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

In an embodiment, the nutritional compositions include an amino acid selected from the group consisting of alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, ornithine, tryptophan, tyrosine, valine, or combinations thereof. In an embodiment, the amino acid is citrulline. In an embodiment, the amino acid is a branched chain amino acid selected from the group consisting of isoleucine, leucine, valine, or combinations thereof.

In an embodiment, the nutritional compositions include an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

In an embodiment, the nutritional compositions include a vitamin selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, choline or combinations thereof.

In an embodiment, the nutritional compositions include a mineral selected from the group consisting of boron, calcium, chromium, copper, iodine, iron, magnesium, manganese, molybdenum, nickel, phosphorus, potassium, selenium, silicon, tin, vanadium, zinc, or combinations thereof.

In an embodiment, the nutritional compositions include a compound selected from the group consisting of α-ketoglutarate, L-carnitine, or combinations thereof.

In an embodiment, the nutritional compositions are in a form selected from the group consisting of tablets, capsules, liquids, chewables, soft gels, sachets, powders, syrups, liquid suspensions, emulsions, solutions, or combinations thereof. In an embodiment, the nutritional compositions are in the form of a powder.

In an embodiment, the nutritional compositions are oral nutritional supplements, a tube feeding, or combinations thereof.

In an embodiment, the nutritional compositions are a source of complete nutrition. In another embodiment, the nutritional compositions are a source of incomplete nutrition.

In yet another embodiment, methods for stimulating muscle protein synthesis in an individual in need of same are provided. The methods include administering to the individual a nutritional composition comprising an effective amount of α-hydroxyisocaproic acid and an effective amount of α-ketoglutarate.

In still yet another embodiment, methods for minimizing catabolism of muscle protein in an individual in need of same are provided. The methods include administering to the individual a nutritional composition comprising an effective amount of α-hydroxyisocaproic acid and an effective amount of α-ketoglutarate.

In another embodiment, methods for preserving lean body mass in an individual in need of same are provided. The methods include administering to the individual a nutritional composition comprising an effective amount of α-hydroxyisocaproic acid and an effective amount of α-ketoglutarate.

In yet another embodiment, methods for reducing unloading-induced bone loss in an individual in need of same are provided. The methods include administering to the individual a nutritional composition comprising an effective amount of α-hydroxyisocaproic acid and an effective amount of α-ketoglutarate.

In still yet another embodiment, methods for attenuating skeletal muscle atrophy in an individual in need of same are provided. The methods include administering to the individual a nutritional composition comprising an effective amount of α-hydroxyisocaproic acid and and an effective amount of α-ketoglutarate.

In another embodiment, methods for alleviating a high uremic load in an individual in need of same are provided. The methods include administering to the individual a nutritional composition comprising an effective amount of α-hydroxyisocaproic acid and and an effective amount of α-ketoglutarate.

In an embodiment, the individual is selected from the group consisting of the elderly, those with a medical condition, or combinations thereof.

In an embodiment, the nutritional compositions are administered to the individual so as to provide the individual with about 0.15 to about 10 g per day, preferably from about 2 g to about 10 g per day, more preferably from about 150 mg to about 2.5 g of α-hydroxyisocaproic acid per day. The nutritional compositions may also be administered to the individual so as to provide the individual with about 0.5 g to about 5 g per day, more preferably from about 2 g to 5 g, or preferably about 1.5 g per day.

In an embodiment, the nutritional compositions further includes citrulline. The nutritional compositions may be administered to the individual so as to provide the individual with about 1 g to about 15 g citrulline per day, more preferably from about 2 g to about 15 g of citrulline per day, even more preferably from about 2 g to about 7 g, even more preferably from about 2 g to about 5 g of citrulline per day. The nutritional compositions may also be administered to the individual so as to provide the individual with about 4 g to about 7 g of citrulline per day.

The nutritional compositions includes α-ketoglutarate in a form selected from the group consisting of ornithine α-ketoglutarate, arginine α-ketoglutarate, ketoisocaproic acid (KIC) or combinations thereof. The nutritional compositions may be administered to the individual so as to provide the individual with about 2 g to about 20 g of α-ketoglutarate per day. The nutritional composition may also be administered to the individual so as to provide the individual with about 10 g to about 30 g of α-ketoglutarate per day.

In an embodiment, the nutritional compositions further include eicosapentaenoic acid. The nutritional compositions may be administered to the individual so as to provide the individual with about 0.25 g to about 5 g, more preferably form about 250 mg to about 3 g, even more preferably from about 250 mg to 1.5 g of eicosapentaenoic acid per day.

In an embodiment, the nutritional compositions further include at least one nucleotide selected from the group consisting of a subunit of deoxyribonucleic acid (“DNA”), a subunit of ribonucleic acid (“RNA”), polymeric forms of DNA and RNA, yeast RNA, or combinations thereof. The at least one nucleotide may be an exogenous nucleotide.

In an embodiment, the nutritional compositions further include at least one branched chain amino acid selected from the group consisting of leucine, isoleucine, valine, or combinations thereof.

In an embodiment, the nutritional compositions further include L-carnitine.

An advantage of the present disclosure is to provide improved nutritional compositions.

Another advantage of the present disclosure is to provide nutritional compositions that maximize skeletal muscle protein synthesis.

Another advantage of the present disclosure is to provide nutritional compositions that minimize catabolism of skeletal muscle proteins.

Yet another advantage of the present disclosure is to provide nutritional compositions that preserve lean body mass.

Still yet another advantage of the present disclosure is to provide nutritional compositions that help to improve recovery from physical activity.

Another advantage of the present disclosure is to provide nutritional compositions that help to reduce healthcare costs.

Yet another advantage of the present disclosure is to provide nutritional compositions that help to reduce unloading-induced bone loss.

Still yet another advantage of the present disclosure is to provide nutritional compositions that help to attenuate skeletal muscle atrophy in individuals in need of same.

Another advantage of the present disclosure is to provide nutritional compositions that help to alleviate a high uremic load.

Additional features and advantages are described herein, and will be apparent from the following Detailed Description.

### DETAILED DESCRIPTION

As used herein, “about” is understood to refer to numbers in a range of numerals. Moreover, all numerical ranges herein should be understood to include all integer, whole or fractions, within the range.

As used herein the term α-hydroxyisocaproic acid is understood as also comprising analogs of α-hydroxyisocaproic acid such as keto-isocaproic acid (KIC), for example.

As used herein, the term “amino acid” is understood to include one or more amino acids. The amino acid can be, for example, alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, ornithine or combinations thereof.

As used herein, “animal” includes, but is not limited to, mammals, which include but is not limited to, rodents, aquatic mammals, domestic animals such as dogs and cats, farm animals such as sheep, pigs, cows and horses, and humans. Wherein the terms “animal” or “mammal” or their plurals are used, it is contemplated that it also applies to any animals that are capable of the effect exhibited or intended to be exhibited by the context of the passage.

As used herein, the term “antioxidant” is understood to include any one or more of various substances such as beta-carotene (a vitamin A precursor), vitamin C, vitamin E, and selenium) that inhibit oxidation or reactions promoted by Reactive Oxygen Species (“ROS”) and other radical and non-radical species. Additionally, antioxidants are molecules capable of slowing or preventing the oxidation of other molecules. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

As used herein, “complete nutrition” includes nutritional products and compositions that contain sufficient types and levels of macronutrients (protein, fats and carbohydrates) and micronutrients to be sufficient to be a sole source of nutrition for the animal to which it is being administered to. Patients can receive 100% of their nutritional requirements from such complete nutritional compositions.

As used herein, “effective amount” is an amount that prevents a deficiency, treats a disease or medical condition in an individual or, more generally, reduces symptoms, manages progression of the diseases or provides a nutritional, physiological, or medical benefit to the individual. A treatment can be patient- or doctor-related.

While the terms “individual” and “patient” are often used herein to refer to a human, the invention is not so limited. Accordingly, the terms “individual” and “patient” refer to any animal, mammal or human having or at risk for a medical condition that can benefit from the treatment.

As used herein, sources of ω-3 fatty acids include, for example, fish oil, krill, plant sources of ω-3, flaxseed, canola oil, walnut, and algae. Examples of ω-3 fatty acids include, for example, α-linolenic acid (“ALA”), docosahexaenoic acid (“DHA”), stearidonic acid (SDA), eicosapentaenoic acid (“EPA”), or combinations thereof.

As used herein, “food grade micro-organisms” means micro-organisms that are used and generally regarded as safe for use in food.

As used herein, “incomplete nutrition” includes nutritional products or compositions that do not contain sufficient levels of macronutrients (protein, fats and carbohydrates) or micronutrients to be sufficient to be a sole source of nutrition for the animal to which it is being administered to. Partial or incomplete nutritional compositions can be used as a nutritional supplement.

As used herein, “long term administrations” are preferably continuous administrations for more than 6 weeks. Alternatively, “short term administrations,” as used herein, are continuous administrations for less than 6 weeks.

As used herein, “mammal” includes, but is not limited to, rodents, aquatic mammals, domestic animals such as dogs and cats, farm animals such as sheep, pigs, cows and horses, and humans. Wherein the term “mammal” is used, it is contemplated that it also applies to other animals that are capable of the effect exhibited or intended to be exhibited by the mammal.

The term “microorganism” is meant to include the bacterium, yeast and/or fungi, a cell growth medium with the microorganism, or a cell growth medium in which microorganism was cultivated.

As used herein, the term “minerals” is understood to include boron, calcium, chromium, copper, iodine, iron, magnesium, manganese, molybdenum, nickel, phosphorus, potassium, selenium, silicon, tin, vanadium, zinc, or combinations thereof.

As used herein, a “non-replicating” microorganism means that no viable cells and/or colony forming units can be detected by classical plating methods. Such classical plating methods are summarized in the microbiology book: James Monroe Jay, et al., “Modern food microbiology,” 7th edition, Springer Science, New York, N.Y. p. 790 (2005). Typically, the absence of viable cells can be shown as follows: no visible colony on agar plates or no increasing turbidity in liquid growth medium after inoculation with different concentrations of bacterial preparations (‘non replicating’ samples) and incubation under appropriate conditions (aerobic and/or anaerobic atmosphere for at least 24 h). For example, bifidobacteria such as Bifidobacterium longum, Bifidobacterium lactis and Bifidobacterium breve or lactobacilli, such as Lactobacillus paracasei or Lactobacillus rhamnosus, may be rendered non-replicating by heat treatment, in particular low temperature/long time heat treatment.

As used herein, “normal bone growth” refers to the process by which childhood and adolescent bones are sculpted by modeling, which allows for the formation of new bone at one site and the removal of old bone from another site within the same bone. This process allows individual bones to grow in size and to shift in space. During childhood bones grow because resorption (the process of breaking down bone) occurs inside the bone while formation of new bone occurs on its outer (periosteal) surface. At puberty the bones get thicker because formation can occur on both the outer and inner (endosteal) surfaces. The remodeling process occurs throughout life and becomes the dominant process by the time that bone reaches its peak mass (typically by the early 20s). In remodeling, a small amount of bone on the surface of trabeculae or in the interior of the cortex is removed and then replaced at the same site. The remodeling process does not change the shape of the bone, but it is nevertheless vital for bone health. Modeling and remodeling continue throughout life so that most of the adult skeleton is replaced about every 10 years. While remodeling predominates by early adulthood, modeling can still occur particularly in response to weakening of the bone.

As used herein, a “nucleotide” is understood to be a subunit of deoxyribonucleic acid (“DNA”), ribonucleic acid (“RNA”), polymeric RNA, polymeric DNA, or combinations thereof. It is an organic compound made up of a nitrogenous base, a phosphate molecule, and a sugar molecule (deoxyribose in DNA and ribose in RNA). Individual nucleotide monomers (single units) are linked together to form polymers, or long chains. Exogenous nucleotides are specifically provided by dietary supplementation. The exogenous nucleotide can be in a monomeric form such as, for example, 5′-Adenosine Monophosphate (“5′-AMP”), 5′-Guanosine Monophosphate (“5′-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”), or combinations thereof. The exogenous nucleotide can also be in a polymeric form such as, for example, an intact RNA. There can be multiple sources of the polymeric form such as, for example, yeast RNA.

“Nutritional products,” or “nutritional compositions,” as used herein, are understood to include any number of optional additional ingredients, including conventional food additives (synthetic or natural), for example one or more acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount. The nutritional products or compositions may be a source of complete nutrition or may be a source of incomplete nutrition.

As used herein the term “patient” is understood to include an animal, especially a mammal, and more especially a human that is receiving or intended to receive treatment, as it is herein defined.

As used herein, “phytochemicals” or “phytonutrients” are non-nutritive compounds that are found in many foods. Phytochemicals are functional foods that have health benefits beyond basic nutrition, are health promoting compounds that come from plant sources, and may be natural or purified. “Phytochemicals” and “Phytonutrients” refers to any chemical produced by a plant that imparts one or more health benefit on the user. Non-limiting examples of phytochemicals and phytonutrients include those that are:

i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3-gallate, theaflavin, theaflavin-3-gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (formononetin), genistein (biochanin A), glycitein), dihydroflavonols, chalcones, coumestans (phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins;

ii) terpenes (isoprenoids) which include carotenoids (tetraterpenoids) including carotenes (such as, for example, α-carotene, β-carotene, γ-carotene, δ-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta sitosterol, gamma sitosterol, stigmasterol), tocopherols (vitamin E), and omega-3, 6, and 9 fatty acids (such as, for example, gamma-linolenic acid); triterpenoid (such as, for example, oleanolic acid, ursolic acid, betulinic acid, moronic acid);

iii) betalains which include Betacyanins (such as: betanin, isobetanin, probetanin, neobetanin); and betaxanthins (non glycosidic versions) (such as, for example, indicaxanthin, and vulgaxanthin);

iv) organosulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide;

v) protein inhibitors, which include, for example, protease inhibitors;

vi) other organic acids which include oxalic acid, phytic acid (inositol hexaphosphate); tartaric acid; and anacardic acid; or

vii) combinations thereof.

As used in this disclosure and the appended claims, the singular forms “a,” “an” and “the” include plural referents unless the context clearly dictates otherwise. Thus, for example, reference to “a polypeptide” includes a mixture of two or more polypeptides, and the like.

As used herein, a “prebiotic” is a food substance that selectively promotes the growth of beneficial bacteria or inhibits the growth or mucosal adhesion of pathogenic bacteria in the intestines. They are not inactivated in the stomach and/or upper intestine or absorbed in the gastrointestinal tract of the person ingesting them, but they are fermented by the gastrointestinal microflora and/or by probiotics. Prebiotics are, for example, defined by Glenn R. Gibson and Marcel B. Roberfroid, “Dietary Modulation of the Human Colonic Microbiota: Introducing the Concept of Prebiotics,” J. Nutr., 125: 1401-1412 (1995). Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof.

As used herein, probiotic micro-organisms (hereinafter “probiotics”) are food-grade microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See, Salminen S, Ouwehand A. Benno Y. et al., “Probiotics: how should they be defined?,” Trends Food Sci. Technol., 10, 107-10 (1999). In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

The terms “protein,” “peptide,” “oligopeptides” or “polypeptide,” as used herein, are understood to refer to any composition that includes, a single amino acids (monomers), two or more amino acids joined together by a peptide bond (dipeptide, tripeptide, or polypeptide), collagen, precursor, homolog, analog, mimetic, salt, prodrug, metabolite, or fragment thereof or combinations thereof. For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring amino acids, and that many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present invention include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (“GPI”) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide.

Non-limiting examples of proteins include dairy based proteins, plant based proteins, animal based proteins and artificial proteins. Dairy based proteins may be selected from the group consisting of casein, micellar casein, caseinates, casein hydrolysate, whey, whey hydrolysates, whey concentrates, whey isolates, milk protein concentrate, milk protein isolate, or combinations thereof. Plant based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, and any proteins derived from beans, buckwheat, lentils, pulses, single cell proteins, or combinations thereof. Animal based proteins may be selected from the group consisting of beef, poultry, fish, lamb, seafood, or combinations thereof.

All dosage ranges contained within this application are intended to include all numbers, whole or fractions, contained within said range.

As used herein, a “synbiotic” is a supplement that contains both a prebiotic and a probiotic that work together to improve the microflora of the intestine.

As used herein, the terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, such as nitrogen imbalance or muscle loss. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition.

As used herein, a “tube feed” is a complete or incomplete nutritional product or composition that is administered to an animal's gastrointestinal system, other than through oral administration, including but not limited to a nasogastric tube, orogastric tube, gastric tube, jejunostomy tube (“J-tube”), percutaneous endoscopic gastrostomy (“PEG”), port, such as a chest wall port that provides access to the stomach, jejunum and other suitable access ports.

As used herein the term “vitamin” is understood to include any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e. MK-4, MK-7), folic acid and biotin) essential in minute amounts for normal growth and activity of the body and obtained naturally from plant and animal foods or synthetically made, pro-vitamins, derivatives, choline, analogs.

The present disclosure is related to nutritional compositions having a combination of nutrients and food ingredients to maximize muscle protein synthesis while minimizing the catabolism of muscle proteins such that the lean body mass of patients including, for example, the elderly and those with illness is preserved as well as possible.

The nutritional compositions of the present disclosure include α-hydroxycaproic acid (“HICA”) in combination with other compounds to maximize the anabolism and minimize the catabolism of muscle tissue. Applicant has found that various combinations with α-HICA deliver superior benefits due to better taste profile (improving compliance and therefore efficacy) as well as complementary metabolic benefits. For example, α-HICA is a leucine metabolite with anabolic benefits directly related to protein synthesis while other compounds such as citrulline deliver benefits ancillary to the anabolic process.

The translational control of skeletal muscle protein synthesis includes control points at initiation, elongation and termination. In addition to the step in translation initiation involving the binding of messenger ribonucleic acid (“mRNA”) to the 40S ribosomal subunit, regulation can occur through modulation of the binding of the initiator methionyl-tRNA (“met-tRNAi”) to the 40S ribosomal subunit to form the 43S preinitiation complex. In this step, the eIF2-GTP-met-tRNAi complex binds to the 40S ribosomal subunit to form a ternary complex. The guanosine triphosphate (“GTP”) bound to eIF2 is subsequently hydrolyzed to guanosine diphosphate (“GDP”), and the eIF2-GDP complex is released from the 40S ribosomal subunit. eIF2 must then exchange GDP for GTP in order to participate in a subsequent round of initiation and form a new ternary complex. A second translation initiation factor, eIF2B, mediates the guanine nucleotide exchange on eIF2 and the inhibition of eIF2B activity reduces the amount of eIF2-GTP available for ternary complex formation. In part, the activity of eIF2B is regulated by phosphorylation of the α-subunit of eIF2, which becomes a competitive inhibitor of eIF2B when phosphorylated on the α-subunit. Moreover, α-HICA mediates its acute effects on global protein synthesis via enhanced translational efficiency through increased eIF2B activity and ternary complex formation.

The nutritional compositions of the present disclosure may be provided to an individual or patient in one bolus, or in several smaller doses. However, the nutritional compositions of the present disclosure should provide the individual with an amount of α-HICA ranging from about 0.15 to about 10 g per day, preferably from about 2 g to about 10 g per day, more preferably from about 150 mg to about 2.5 g of α-hydroxyisocaproic acid per day. In an embodiment, the individual is provided with about 0.5 g to about 5 g per day, more preferably from about 2 g to 5 g, even more preferably 1.5 g α-HICA per day.

In an embodiment, the nutritional compositions are administered to the individual so as to provide the individual with about [symbol] The nutritional compositions may also be administered to the individual so as to provide the individual with about

[symbol]

In an embodiment, nutritional compositions of the present disclosure include α-HICA and citrulline. Citrulline is a non-protein amino acid that is found in significant dietary amounts only in watermelon (Citrullus lanatus). Intake of citrulline can lead to formation of polyamines. Polyamines such as agmatine, putrescine, spermidine and spermine have been reported to be involved in a variety of physiological and biochemical phenomena including upregulation of protein kinase C (“PKC”), extracellular signal-regulated kinase (“ERK”), and transforming growth factor-beta1 (“TGF-beta1”).

The metabolic fate of citrulline is conversion to arginine. In fact, citrulline is very effective in raising serum arginine, which is a source of nitric oxide (“NO”) in the body. NO is important for relaxation of blood vessels and delivery of blood flow to tissues in the body. With improved blood flow, nutrients and other compounds in the blood can be delivered more efficiently to the skeletal muscle tissues. Further, NO is an anabolic signal as well as a facilitator for stimulation of protein synthesis and release of growth factors such as polyamines mentioned above. NO also leads to release of insulin and IGF-1 leading to increased uptake of anabolic substrates as well as bio-utlization of the substrates.

Guadagni and Biolo indicate that additional protein may be needed in individuals with inflammation (such as the elderly or individuals with illness) in part to maintain the levels of arginine and glutamine. See, Guadagni and Biolo, Effects of inflammation and/or inactivity on the need for dietary protein, Volume 12, Issue 6, p. 617-622 (2009). Citrulline can serve to maintain arginine levels. Additionally, it can help to maintain glutamine levels since glutamine conversion to citrulline in the small intestine will be reduced by a feedback signal from the citrulline provided exogenously. This will reduce the need for muscle catabolism to provide arginine and glutamine for bodily functions.

It is further possible that the combination of α-HICA and citrulline will synergistically improve the maintenance of lean body mass in elderly that do a limited amount of exercise and/or physical therapy. Citrulline has been shown to have an anabolic effect in malnourished aged animals. The anabolic signal in the elderly population is typically down-regulated. The addition of both α-HICA and citrulline will provide a strong boost to this signal. This improved recovery from physical activity will allow for accelerated recovery from forced inactivity due to illness or trauma. A reduction in cost of care could also be realized based on reduced number physical therapy sessions and a faster return to full independent living and a return to work.

As mentioned above, the nutritional compositions of the present disclosure may be provided to an individual or patient in one bolus, or in several smaller doses. However, the nutritional compositions of the present disclosure should provide the individual with an amount of citrulline ranging from about 1 g to about 15 g citrulline per day, more preferably from about 2 g to about 15 g of citrulline per day, even more preferably from about 2 g to about 7 g, even more preferably from about 2 g to about 5 g of citrulline per day. In an embodiment, the individual is provided with from about 4 g to about 7 g citrulline per day.

The nutritional compositions of the present disclosure may also include a synergistic combination of α-HICA and α-ketoglutarate (“AKG”), a precursor of glutamine. In a piglet model where the piglet was stressed with lipopolysaccharide (“LPS”) administration, AKG increased phosphorylation of intestinal mammalian target of rapamycin (“mTOR”) leading to increased protein synthesis and anti-inflammatory responses. Also, AKG increased villous height and reduced crypt depth, and therefore, the potential to increase absorptive capacity (increased absorption of amino acids). Applicant has found that these potential benefits of nutritional compositions including α-HICA and AKG (e.g., oxidative damage, absorption) may lead to increased nutrient delivery leading to further anabolism particularly in inflammatory conditions.

As mentioned above, the nutritional compositions of the present disclosure may be provided to an individual or patient in one bolus, or in several smaller doses. However, the nutritional compositions of the present disclosure should provide the individual with an amount of AKG ranging from about 2 g to about 20 g of α-ketoglutarate per day or from about 10 g to about 30 g per day. The AKG may be in the form of ornithine AKG, arginine AKG, or combinations thereof.

The addition of exogenous nucleotides can make the AKG more effective by two mechanisms: (i) the maintenance of AKG levels by reducing the use of glutamine to make nucleotides in the intestinal tract, and (ii) the enhanced maintenance of villous height as shown in previous studies with nucleotides. The intestinal health provided by nucleotides is especially important in the elderly due to malnutrition or just the reduced general anabolism associated with increased age.

Branched chain amino acids (“BCAA”), are known to be indispensible amino acids. BCAAs, along with other indispensible amino acids, must be provided exogenously to allow for muscle protein synthesis. BCAAs, especially leucine, also serve as signaling molecules to stimulate muscle protein synthesis. This can be via two mechanisms. The first mechanism is stimulation of insulin release since leucine is a strong secretagogue. The second mechanism is more direct as leucine can stimulate the eukaryotic inducing factor that turns on muscle protein synthesis.

It is important to provide all three BCAAs (i.e., leucine, isoleucine, and valine) in any formulation since the large increase of one BCAA can cause a relative deficiency of the other two BCAAs. As BCAAs are known for their undesirable sensory profile, addition of analogs such as α-HICA as well as designer, or high quality, proteins such as, for example, whey protein micelles is a effective way of delivering the benefit while improving patient compliance and therefore clinical outcome leading to better quality of life as well as health economic advantages. Further, combinations with immunomodulating agents such as lactowolfberry can bring synergistic benefits to the patient with low graded inflammation, suppressed anabolism and immunosenescence (e.g., elderly, or those with illness).

In another embodiment, nutritional compositions of the present disclosure may include α-HICA and an ω-3 fatty acid. Example of ω-3 fatty acids include, for example, docosahexaenoic acid (“DHA”), eicosapentaenoic acid (“EPA”) and α-linolenic acid (“ALA”). EPA, an omega-3 polyunsaturated fatty acid, has been shown to attenuate skeletal muscle atrophy in cancer cachexia as well as sepsis and to reduce unloading-induced bone loss through a common cellular signaling pathway by minimizing activation of nuclear factor-κβ (“NF-κβ”). Applicant has found that nutritional compositions having α-HICA and EPA synergistically impact musculoskeletal health through both an attenuated loss of lean body mass and bone mineral density through targeted inhibition of NFkβ. Further, α-HICA and EPA can enhance skeletal muscle protein synthesis (as mediated through the mTOR pathway) and reduce endogenous muscle proteolysis (as mediated through the ubiquitin-proteasome pathway), respectively, under catabolic, disuse or aging conditions. The nutritional therapy will result in preserved lean body mass, which will provide tonic loading to the underlying bone and act as an osteogenic stimulus for bone turnover and minimize fracture risk.

Improved preservation of lean body mass will help to maintain metabolic homeostasis and functional mobility. Further the preservation of bone mass density will reduce the risk of fracture thus leading to improved quality of life as well as healthcare cost savings.

The nutritional compositions of the present disclosure may be provided to an individual or patient in one bolus, or in several smaller doses. However, the nutritional compositions of the present disclosure should provide the individual with an amount of EPA ranging from about 0.25 g to about 5 g, more preferably form about 250 mg to about 3 g, even more preferably from about 250 mg to 1.5 g of eicosapentaenoic acid per day. In an embodiment, the individual is provided with about 750 mg of EPA per day.

The delivery and bioavailability of nutritional compositions having α-HICA and EPA can be improved by (i) packaging (e.g., providing a UV-barrier and/or O₂ scavenging inner layers); (ii) manufacturing (e.g., providing aseptic production, reducing “head space,” and reducing heat exposure), and (iii) encapsulation of a lipid emulsion containing both α-HICA and EPA (e.g., protecting the composition during manufacturing and initial digestion). Further, a vegetarian source of EPA can provide a sustainable source of long-chain polyunsaturated fatty acids (“LC-PUFA”) with improved organoleptic properties.

The nutritional compositions of the present disclosure may provide effective amounts of α-HICA to prevent muscle wasting. Muscle wasting is commonly noted in individuals with chronic kidney disease. Applicant has found, however, that the application of α-HICA to the kidney disease patient segment has several benefits. For example, administering nutritional compositions having α-HICA to the kidney disease patient segment may provide nitrogen or protein sparing effects and improve nitrogen balance in chronic renal failure especially in patients displaying uremia. Branched chain α-keto acids, and α-HICA can take up amine groups from the elevated nitrogenous environment of the uremic patient and thus reduce the overall nitrogenous load. This substitution also partly reduces the total protein intake by patients thereby reducing a further increase in the nitrogen load in uremia patients both of which ameliorate the toxicity associated with elevated urea levels. Providing a portion of the protein needs via substitution with α-HICA and/or other keto-acids may improve the total protein intake of the patient that may support muscle protein.

Further, α-HICA like its precursor leucine, may stimulate muscle protein synthesis and/or limit muscle protein breakdown beneficial for this patient population. U.S. Pat. No. 4,752,619 supports the use of the aforementioned products in conjunction with 20-30 g/day mixed quality protein diet, and a vitamin and mineral supplement.

Applicant has also surprisingly found that nutritional compositions of the present disclosure having a combination of α-HICA and L-carnitine demonstrate synergistic effects in chronic kidney patients, and especially in patients suffering from uremia. L-carnitine is a quaternary ammonium compound biosynthesized from the amino acids lysine and methionine in the liver and kidney. It is found to be deficient in kidney disease owing to impaired biosynthesis, reduced protein intake and losses via dialysis in dialyzed patients. The benefits of L-carnitine supplementation in kidney disease patients may include improvement in erythropoietin-resistant anemia, muscle symptoms, cardiac performance and functional capacities, benefits that may also support muscle function. A combination of α-HICA and L-carnitine will offer the dual benefit of alleviating the uremic load to an extent while providing the deficient product L-carnitine that may support muscle function at least in part, due to its primary function as a transporter of long chain fatty acids to the mitochondria for energy-yielding oxidation.

Existing nutrition support solutions for elderly and patients that have insufficient muscle anabolism and excessive muscle catabolism are lacking in effectiveness. Furthermore, in elderly individuals, there is significant lean body mass loss leading to loss of independence, functionality and quality of life. Further, there is a decline in cognitive ability in elderly patients, and the healthcare costs associated with these morbidities are high. The traditional response to loss of lean body mass has been to provide an increased level of protein to the patient.

Applicant has found that the use of additional beneficial ingredients allows for more efficient use of the administered protein for preservation of lean body mass. Thus, the nutritional compositions of the present disclosure improve the preservation of lean body mass in elderly individuals or patients at risk of muscle loss (e.g. sarcopenia, cachexia, immobilization) by increasing muscle anabolism while simultaneously reducing muscle catabolism. The ingredients that provide the benefit of increased anabolism and decreased catabolism and can be incorporated into both oral supplements and complete feeding products suitable for complete feeding by either oral or tube feeding administration. The nutritional compositions of the present disclosure can also be assembled and packaged as powders for dissolution at the time of use.

In an embodiment wherein the nutritional compositions are oral supplements, the supplements may contain active ingredients plus an appropriate nutritional profile that contains 10 or more grams of high quality protein, which may be provided as whey protein micelle, lipids with the EPA and DHA, and carbohydrates for energy and palatability. Vitamins such as vitamin D and minerals and ingredients such as lactowolfberry, and nucleotides may also be included.

Complete feeding products may have all of the nutrients necessary to support life plus the active ingredients necessary for increased anabolism and decreased catabolism (e.g., α-HICA and/or other beneficial ingredients such as L-carnitine, citrulline, AKG, EPA, etc.).

The nutritional compositions of the present disclosure may be administered by any means suitable for human administration, and in particular for administration in any part of the gastrointestinal tract. Enteral administration, oral administration, and administration through a tube or catheter are all covered by the present disclosure. The nutritional compositions may also be administered by means selected from oral, rectal, sublingual, sublabial, buccal, topical, etc.

If the nutritional compositions are formulated to be administered orally, the compositions may be a liquid oral nutritional supplement (e.g., incomplete feeding) or a complete feeding. In this manner, the nutritional compositions may be administered in any known form including, for example, tablets, capsules, liquids, chewables, soft gels, sachets, powders, syrups, liquid suspensions, emulsions and solutions in convenient dosage forms. In soft capsules, the active ingredients are preferably dissolved or suspended in suitable liquids, such as fatty oils, paraffin oil or liquid polyethylene glycols. Optionally, stabilizers may be added.

Suitable nutritional composition formats according to the present disclosure include, for example, infant formulas, solutions, ready-for-consumption compositions (e.g. ready-to-drink compositions or instant drinks), liquid comestibles, soft drinks, juice, sports drinks, milk drinks, milk-shakes, yogurt drinks, soup, etc. In a further embodiment, the nutritional compositions may be manufactured and sold in the form of a concentrate, a powder, or granules (e.g. effervescent granules), which are diluted with water or other liquid, such as milk or fruit juice, to yield a ready-for-consumption composition (e.g. ready-to-drink compositions or instant drinks).

The nutritional compositions may also include a source of ω-3 and/or ω-6 fatty acids. Examples of sources of ω-3 fatty acids include, for example, fish oil, krill, plant sources of ω-3, flaxseed, walnut, and algae. Non-limiting examples of ω-3 fatty acids include α-linolenic acid (“ALA”), docosahexaenoic acid (“DHA”), and eicosapentaenoic acid (“EPA”). Non-limiting examples of ω-6 fatty acids include linoleic acid (“LA”), arachidonic acid (“ARA”).

In a prefered embodiment, the ω-3 fatty acids are provided in an amount of about 0.25 g to 5.0 g per day, preferably about 1.0 to 3.0 g per day.

In an embodiment, the nutritional compositions include a source of phytochemicals. Phytochemicals are non-nutritive compounds that are found in many fruits and vegetables, among other foods. There are thousands of phytochemicals that can be categorized generally into three main groups. The first group is flavonoids and allied phenolic and polyphenolic compounds. The second group is terpenoids, e.g., carotenoids and plant sterols. The third group is alkaloids and sulfur containing compounds. Phytochemicals are active in the body and, in general, act similarly to antioxidants. They also appear to play beneficial roles in inflammatory processes, clot formation, asthma, and diabetes.

In an embodiment, the nutritional compositions include a source of protein. The protein source may be dietary protein including, but not limited to animal protein (such as milk protein, meat protein or egg protein), vegetable protein (such as soy protein, wheat protein, rice protein, and pea protein), or combinations thereof. In an embodiment, the protein is selected from the group consisting of whey, chicken, corn, caseinate, wheat, flax, soy, carob, pea or combinations thereof.

In an embodiment, vegetable proteins will be included to further enhance the net alkaline profile of the formula and increase the variety of macronutrient sources. Based on the nutritional profile of specific vegetable proteins (e.g., pea protein isolate) there are limitations in the amount of vegetable protein sources that can be included in a formula. For example, the amino acid profile of pea protein includes all of the indispensable amino acids. Pea protein is relatively rich in arginine, but limiting in the sulphur-containing amino acids, methionine, and cysteine. However, it is possible, for example, to blend pea protein isolates with a complete protein source (such as milk protein or complete vegetable proteins) having sufficient sulphur-containing amino acids to offset such deficiency. Canola protein (i.e., isolates, hydrosylates and concentrates) is one such vegetable protein which can provide appreciable amounts of sulfur-containing amino acids to further augment the amino acid profile to deliver the necessary protein quality to the patient. Additionally, animal derived proteins are typically more abundant in sulphur-containing amino acids than vegetable proteins.

The nutritional compositions of the present disclosure may also include a source of carbohydrates. Any suitable carbohydrate may be used in the present nutritional compositions including, but not limited to, sucrose, lactose, glucose, fructose, corn syrup solids, maltodextrin, modified starch, amylose starch, tapioca starch, corn starch or combinations thereof.

The nutritional compositions may also include grains. The grains may include, for example, whole grains, which may be obtained from different sources. The different sources may include semolina, cones, grits, flour and micronized grain (micronized flour), and may originate from a cereal or a pseudo-cereal. In an embodiment, the grain is a hydrolyzed whole grain component. As used herein, a “hydrolyzed whole grain component” is an enzymatically digested whole grain component or a whole grain component digested by using at least an α-amylase, which α-amylase shows no hydrolytic activity towards dietary fibers when in the active state. The hydrolyzed whole grain component may be further digested by the use of a protease, which protease shows no hydrolytic activity towards dietary fibers when in the active state. The hydrolyzed whole grain component may be provided in the form of a liquid, a concentrate, a powder, a juice, a puree, or combinations thereof.

A source of fat may also be included in the present nutritional compositions. The source of fat may include any suitable fat or fat mixture. For example, the fat source may include, but is not limited to, vegetable fat (such as olive oil, corn oil, sunflower oil, high-oleic sunflower, flax seed oil, rapeseed oil, canola oil, high oleic canola oil, hazelnut oil, soy oil, palm oil, coconut oil, blackcurrant seed oil, borage oil, lecithins, and the like), animal fats (such as milk fat), or combinations thereof. The source of fat may also be less refined versions of the fats listed above (e.g., olive oil for polyphenol content).

In an embodiment, the nutritional compositions further include one or more prebiotics. Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

The nutritional compositions may further include one or more probiotics. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

One or more amino acids may also be present in the nutritional compositions. Non-limiting examples of amino acids include alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

In a preferred embodiment, glutamine is provided in an amount of about 10 g to 40 g per day.

One or more antioxidants may also be present in the nutritional compositions. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

The nutritional compositions also include fiber or a blend of different types of fiber. The fiber blend may contain a mixture of soluble and insoluble fibers. Soluble fibers may include, for example, fructooligosaccharides, acacia gum, inulin, etc. Insoluble fibers may include, for example, pea outer fiber.

The nutritional compositions of the present disclosure may be a source of either incomplete or complete nutrition. The nutritional compositions may be administered by oral administration or tube feeding. If the nutritional compositions are formulated to be administered orally, the compositions may be a liquid oral nutritional supplement or feeding. The nutritional compositions may also be used for short term or long term tube feeding.

In yet another embodiment, methods of administering the nutritional compositions of the present disclosure are provided. For example, in an embodiment, methods for stimulating muscle protein synthesis in an individual in need of same are provided. In another embodiment, methods for minimizing catabolism of muscle protein in an individual in need of same are provided. In yet another embodiment, methods for preserving lean body mass in an individual in need of same are provided. In still yet another embodiment, methods for reducing unloading-induced bone loss in an individual in need of same are provided. In yet another embodiment, methods for attenuating skeletal muscle atrophy in an individual in need of same are provided. In another embodiment, methods for alleviating a high uremic load in an individual in need of same are provided. The methods include administering to the individual a nutritional composition comprising an effective amount of α-hydroxyisocaproic acid. The nutritional compositions of the present disclosure may also include other active or inactive ingredients as discussed herein above.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A nutritional composition comprising an effective amount of α-hydroxyisocaproic acid and an effective amount of α-ketoglutarate.

2. The nutritional composition according to claim 1, wherein the α-hydroxyisocaproic acid is present in an amount from about 2 g to about 10 g.

3. The nutritional composition according to claim 1, wherein the composition provides an individual with about 0.15 g to about 10 g of α-hydroxyisocaproic acid per day.

4. The nutritional composition according to claim 1, wherein the α-hydroxyisocaproic acid is present in an amount of about 1.5 g.

5. The nutritional composition according to claim 1, wherein the α-ketoglutarate is present in an amount of about 10 g to about 30 g.

6. The nutritional composition according to claim 1, wherein the nutritional composition is administered to the individual so as to provide the individual with about 2 g to 20 g of α-ketoglutarate per day.

7. The nutritional composition according to claim 1, wherein the nutritional composition includes α-ketoglutarate in a form selected from the group consisting of ornithine α-ketoglutarate, arginine α-ketoglutarate, and combinations thereof.

8. The nutritional composition according to claim 1, further comprising a source of ω-3 fatty acids, wherein the source of ω-3 fatty acids is selected from the group consisting of fish oil, krill, plant sources containing ω-3 fatty acids, flaxseed, walnut, algae, and combinations thereof.

9. The nutritional composition according to claim 9, wherein the ω-3 fatty acids are selected from the group consisting of α-linolenic acid (“ALA”), docosahexaenoic acid (“DHA”), stearidonic acid (SDA), eicosapentaenoic acid (“EPA”), and combinations thereof.

10. The nutritional composition according to claim 8, wherein the ω-3 fatty acids are provided in an amount of about 0.25 g to 5.0 g per day, preferably about 1.0 to 3.0 g per day.

11. The nutritional composition according to claim 1, further comprising at least one nucleotide selected from the group consisting of a subunit of deoxyribonucleic acid (“DNA”), a subunit of ribonucleic acid (“RNA”), polymeric forms of DNA and RNA, yeast RNA, and combinations thereof.

12. The nutritional composition according to claim 11, wherein the at least one nucleotide is an exogenous nucleotide.

13. The nutritional composition according to claim 11, wherein the nucleotide is provided in an amount of about 0.5 g to 3 g per day.

14. The nutritional composition according to claim 1, further comprising a phytonutrient selected from the group consisting of flavanoids, allied phenolic compounds, polyphenolic compounds, terpenoids, alkaloids, sulphur-containing compounds, and combinations thereof.

15. The nutritional composition according to claim 14, wherein the phytonutrient is selected from the group consisting of carotenoids, plant sterols, quercetin, curcumin, limonin, and combinations thereof.

16. The nutritional composition according to claim 1, further including a source of protein.

17. The nutritional composition according to claim 14, wherein the source of protein provides the nutritional composition with at least 10 g of high quality protein.

18. The nutritional composition according to claim 14, wherein the source of protein provides an individual with at least 10 g of high quality protein per day.

19. The nutritional composition according to claim 16, wherein the source of protein is selected from the group consisting of dairy based proteins, plant based proteins, animal based proteins, artificial proteins, and combinations thereof.

20. The nutritional composition according to claim 19, wherein the dairy based proteins are selected from the group consisting of casein, micellar casein, caseinates, casein hydrolysate, whey, whey hydrolysates, whey concentrates, whey isolates, whey protein micelles, milk protein concentrate, milk protein isolate, and combinations thereof.

21. The nutritional composition according to claim 19, wherein the plant based proteins are selected from the group consisting of soy protein, pea protein, canola protein, wheat and fractionated wheat proteins, corn proteins, zein proteins, rice proteins, oat proteins, potato proteins, peanut proteins, green pea powder, green bean powder, spirulina, proteins derived from vegetables, beans, buckwheat, lentils, pulses, single cell proteins, and combinations thereof.

22. The nutritional composition according to claim 1, further comprising a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, and combinations thereof.

23. The nutritional composition according to claim 1, further comprising a probiotic selected from the group consisting of Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, and combinations thereof.

24. The nutritional composition according to claim 1, further comprising an amino acid selected from the group consisting of alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, and combinations thereof.

25. The nutritional composition according to claim 24, wherein the amino acid is a branched chain amino acid selected from the group consisting of isoleucine, leucine, valine, and combinations thereof.

26. The nutritional composition according to claim 1, further comprising an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, and combinations thereof.

27. The nutritional composition according to claim 1, further comprising a vitamin selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, and combinations thereof.

28. The nutritional composition according to claim 1, further comprising a mineral selected from the group consisting of boron, calcium, chromium, copper, iodine, iron, magnesium, manganese, molybdenum, nickel, phosphorus, potassium, selenium, silicon, tin, vanadium, zinc, and combinations thereof.

29. The nutritional composition according to claim 1, further including L-carnitine.

30. The nutritional composition according to claim 1, wherein the nutritional composition is in a form selected from the group consisting of tablets, capsules, liquids, chewables, soft gels, sachets, powders, syrups, liquid suspensions, emulsions, solutions, and combinations thereof.

31. The nutritional composition according to claim 1, wherein the nutritional composition is an oral nutritional supplement or a tube feeding.

32. The nutritional composition according to claim 1, wherein the nutritional composition is a source of complete nutrition or of incomplete nutrition.

33. A method selected from the group consisting of:
i) stimulating muscle protein synthesis in an individual in need of same,
ii) minimizing catabolism of muscle protein in an individual in need of same,
iii) preserving lean body mass in an individual in need of same,
iv) reducing unloading-induced bone loss in an individual in need of same,
v) attenuating skeletal muscle atrophy in an individual in need of same, and
vi) alleviating a high uremic load in an individual in need of same comprising administering to the individual a nutritional composition comprising an effective amount of α-hydroxyisocaproic acid and an effective amount of α-ketoglutarate.

34. The nutritional composition according to claim 33, wherein the individual is selected from the group consisting of the elderly, those with a medical condition, and combinations thereof.

35. The nutritional composition according to claim 34, wherein the elderly includes those at risk of disability due to sarcopenia, frailty.

36. The nutritional composition according to claim 33, wherein the nutritional composition is administered to the individual so as to provide the individual with about 150 mg to about 2.5 g of α-hydroxyisocaproic acid per day.

37. The nutritional composition according to claim 33, wherein the nutritional composition is administered to the individual so as to provide the individual with about 0.15 g to about 10 g of α-hydroxyisocaproic acid per day.

38. The nutritional composition according to claim 33, wherein the nutritional composition is administered to the individual so as to provide the individual with about 150 mg to about 2.5 g of α-hydroxyisocaproic acid per day.

39. The nutritional composition according to claim 33, wherein the nutritional composition is administered to the individual so as to provide the individual with about 10 g to about 30 g of α-ketoglutarate per day.

40. The nutritional composition according to claim 33, wherein the nutritional composition is administered to the individual so as to provide the individual with about 2 g to 20 g of α-ketoglutarate per day.

41. The nutritional composition according to claim 1, wherein the nutritional composition includes α-ketoglutarate in a form selected from the group consisting of ornithine α-ketoglutarate, arginine α-ketoglutarate, and combinations thereof.

42. The nutritional composition according to claim 1, wherein the nutritional composition further includes citrulline.

43. The nutritional composition according to claim 42, wherein the nutritional composition is administered to the individual so as to provide the individual with about 2 g to about 15 g of citrulline per day.

44. The nutritional composition according to claim 42, wherein the nutritional composition is administered to the individual so as to provide the individual with about 1 g to about 15 g citrulline per day.

45. The nutritional composition according to claim 1, further comprising a source of ω-3 fatty acids, wherein the source of ω-3 fatty acids is selected from the group consisting of fish oil, krill, plant sources containing ω-3 fatty acids, flaxseed, walnut, algae, and combinations thereof.

46. The nutritional composition according to claim 45, wherein the ω-3 fatty acids are selected from the group consisting of α-linolenic acid (“ALA”), docosahexaenoic acid (“DHA”), stearidonic acid (SDA), eicosapentaenoic acid (“EPA”), and combinations thereof.

47. The nutritional composition according to claim 45, wherein the ω-3 fatty acids are provided in an amount of about 0.25 g to 5.0 g per day.

48. The nutritional composition according to claim 46, wherein the nutritional composition is administered to the individual so as to provide the individual with about 0.25 g to about 5 g.

49. The nutritional composition according to claim 46, wherein the nutritional composition is administered to the individual so as to provide the individual with about 250 mg to about 1.5 g of eicosapentaenoic acid per day.

50. The nutritional composition according to claim 1, wherein the nutritional composition further includes at least one nucleotide selected from the group consisting of a subunit of deoxyribonucleic acid (“DNA”), a subunit of ribonucleic acid (“RNA”), polymeric forms of DNA and RNA, yeast RNA, and combinations thereof.

51. The nutritional composition according to claim 50, wherein the at least one nucleotide is an exogenous nucleotide.

52. The nutritional composition according to claim 1, wherein the nutritional composition further includes at least one branched chain amino acid selected from the group consisting of leucine, isoleucine, valine, and combinations thereof.

53. The nutritional composition according to claim 1, wherein the nutritional composition further includes L-carnitine.
